SEQUENCE LISTING (3 sequences)

SEQ ID NO: 1 - DNA, 21 bp, Artificial sequence
  PCR primer
tagccatgcccttagtaggac

SEQ ID NO: 2 - DNA, 21 bp, Artificial Sequence
  PCR primer
gacgactaccctgtactcagg

SEQ ID NO: 3 - DNA, 27 bp, Artificial Sequence
  PCR primer
aacagtggtgagttcgttggatggctt

(19) United States
(12) Patent Application Publication
(10) Pub. No.: US 2009/0010880 A1
(43) Pub. Date: Jan. 8, 2009

(54) 2-Amino-2,7-dideoxy-alpha-D-glycero-D-gluco-heptopyranosyl Inhibitors of Positive Sense Single-Stranded RNA Envelope Viruses

(75) Inventors: Alexander V. Birk, Doylestown, PA (US); Edward J. Dubovi, Ithica, NY (US); Hazel H. Szeto, New York, NY (US)

Correspondence Address: FOX ROTHSCHILD LLP, 1101 MARKET STREET, SUITE 2600, PHILADELPHIA, PA, 19107-2950, US

(73) Assignee: Institute for Hepatitis and Virus Research, Doylestown, PA (US)

(21) Appl. No.: 12/134,790

(22) Filed: Jun. 6, 2008

Related U.S. Application Data
(60) Provisional application No. 60/933,529, filed on Jun. 7, 2007.

(51) Int. Cl. A61K 38/21; C07D 315/00; A61P 31/00; A61K 31/35

(52) U.S. Cl. 424/85.4; 549/417; 514/459

(57) ABSTRACT

The present invention is directed to compounds, compositions and methods comprising the aminoglycoside moiety represented by Formula II for treating and preventing the spread of positive sense single-stranded RNA envelope viral infections. One embodiment of the present invention uses geneticin or its analogs, including 2-amino-2,7-dideoxy-alpha-D-glycero-D-gluco-heptopyranose, as the antiviral agent. The compounds, compositions and methods of the present invention are applicable to infections resulting from Hepatitis C virus, West Nile virus, Yellow Fever virus, Dengue virus, Bovine Viral Diarrhea virus, Equine Arteritis virus, and/or Sindbis virus.

### CROSS REFERENCE TO RELATED APPLICATIONS

This application claims priority benefit of U.S. Provisional Application No. 60/933,529, filed Jun. 7, 2007.

### FIELD OF THE INVENTION

The present invention is directed to the treatment of viral infections, specifically positive sense single-stranded RNA envelope viral infections.

### BACKGROUND OF THE INVENTION

Positive sense single-stranded RNA envelope viruses, for example, members of Flaviviridae family, present health-related problems to human and animal populations alike. For example, Hepatitis C virus (HCV) is a major cause of chronic liver disease, leading to liver cirrhosis and hepatocellular carcinoma (1). It is a major pathogen infecting about 170 million individuals worldwide (1, 2).

Another example of a positive sense single-stranded RNA envelope virus is Dengue virus. Dengue virus (DV) infections include a spectrum of illnesses caused by infection with one of four serotypes of DV (types 1-4) that occur in many tropical and subtropical regions of the world. The geographic distribution of Dengue has expanded over the last 30 years to include more than 100 countries (WHO (3)). Based on the number of infections with DV (about 50 million per year) and the fact that there are hundreds of thousands of cases of severe Dengue disease each year (WHO, (3)), in 2005, the US Centers for Disease Control (CDC) considered Dengue to be the most important mosquito-borne viral disease affecting humans. Global distribution of DV is comparable to that of malaria, and an estimated 2.5 billion people live in areas at risk for epidemic transmission. Each year hundreds of thousands of cases of Dengue Hemorrhagic Fever (DHF) are reported, reaching a case-fatality rate of about 5%, mostly among children and young adults.

Given the health problems created by these viruses, there is a need to study positive sense single-stranded RNA envelope viruses in order to find suitable means for treating or inhibiting viral infection. One model system for studying such viruses is a replicon. A viral replicon is an RNA molecule, or a region of RNA, that replicates from a single origin of replication. The HCV RNA replicons equipped with a neomycin resistance gene, allow for screening and identification of new inhibitors of HCV intracellular replication. However, since replicons do not undergo a complete replication cycle, drug screening programs and mechanism of action studies based solely on these assays might not identify compounds targeting either early (virion attachment, entry, uncoating) or late (virion assembly, egress) stages of the viral replication cycle. In addition, it is possible that drugs that negatively affect neomycin resistance might also inhibit viral replication of HCV RNA replicons without affecting any of the viral mechanisms, thereby requiring additional screens to filter out the false positives. In this regard, aminoglycosides, for example neomycin analogs, usually are not identified as potential antivirals using such HCV replicon screens.

Given the problems with studying HCV, other viruses may be better models for study. One such model of HCV is bovine viral diarrhea virus (BVDV). BVDV is commonly used as a surrogate model for HCV infection. BVDV is a cytoplasmic positive sense single-stranded RNA envelope virus that belongs to the Flaviviridae family (4). BVDV is known to cause severe lesions in the gastrointestinal tract followed by death in affected animals (5, 6). Two biotypes, cytopathic (cpBVDV) and noncytopathic (ncpBVDV), can be isolated from infected animals (7). However, only cpBVDV induces cytopathology in sensitive cell types, such as Madin-Darby bovine kidney cells (MDBK).

BVDV is a good model of HCV for a variety of reasons. Bovine viral diarrhea virus shares a similar structural organization with hepatitis C virus (8). Like HCV, BVDV may utilize the low-density lipoprotein receptor to enter cells, uses a functionally similar internal ribosome entry site (IRES) for translation, uses an NS4A cofactor with its homologous NS3 protease, possesses a similar NS3 helicase/NTPase as well as a mechanistically similar NS5B RNA-dependent RNA polymerase, and has an apparently equivalent mechanism of virion maturation, assembly and egress (8).

Given the magnitude of the human problems associated with these viruses there is an urgent need for compositions and methods to prevent and/or treat positive sense single-stranded RNA envelope virus infections.

### SUMMARY OF THE INVENTION

One embodiment of the present invention is a compound of Formula II

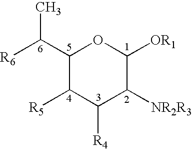

wherein R₁ comprises H, cycloalkyl, carbohydrate, peptide, or nucleotide groups
wherein R₂ and R₃ each independently comprise H, alkyl, cycloalkyl, hydroxyalkyl, alkoxyalkyl, haloalkyl, aryl, arylalkyl, heteroaryl, heteroarylalkyl, alkenyl, alkenylalkyl, alkynyl, alkynylalkyl, acyl, aroyl, heteroaroyl, aminocarbonyl, and alkoxycarbonyl, all optionally substituted with hydroxy, alkoxy, alkyl, haloalkyl, haloalkoxy, amino, alkylamino, dialkylamino, acylamino, alkylthio, alkylsulfoxyl, alkylsulfonyl, cyano, nitro, and/or halogen
wherein R₄, R₅, and R₆ each independently comprise OR₂, halogen, S(O)ₙR₈, CR₉R₁₀R₁₁, CH₂OR, CN, CO₂R, CONR₁₂R₁₃, and NR₁₂R₁₃, wherein R and R₈-R₁₃ are independently selected from the group consisting of H, alkyl, cycloalkyl, alkoxyalkyl, haloalkyl, arylalkyl, heteroarylalkyl, and n=0-2
with the proviso that when R and R₃=H, and R₄, R₅ and R₆=OH, R₁ is other than H.

Preferred compounds include those wherein R₁ is selected from the group consisting of streptamine, or 2-deoxystreptamine, R₁ and R₃ are H, and R₄, R₅, and R₆ are OH.

Another embodiment of the present invention is a composition for treating positive sense single-stranded RNA envelope viral infections comprising the aminoglycoside moiety 2-amino-2,7-dideoxy-alpha-D-glycero-D-gluco-heptopyranose, or an analog thereof. This composition reduces the infectivity of virus particles. The composition is useful for treating viruses selected from the group consisting of Hepatitis C virus (HCV), West Nile virus (WNV), Yellow Fever virus (YFV), Dengue virus (DV), Bovine Viral Diarrhea virus (BVDV), Equine Arteritis virus (EAV), and Sindbis virus (SINV), or a combination of said viruses. The aminoglycoside of the composition may comprise an unmodified 2-amino-2,7-dideoxy-alpha-D-glycero-D-gluco-heptopyranosyl moiety, or an analog thereof. A preferred composition includes geneticin or an analog thereof as the aminoglycoside. Functionalization with modifying groups may be on the amino or hydroxyl groups of the 2-amino-2,7-dideoxy-alpha-D-glycero-D-gluco-heptopyranosyl moiety. The modifying groups are selected from the group consisting of alkyl, cycloalkyl, hydroxyalkyl, alkoxyalkyl, haloalkyl, aryl, arylalkyl, heteroaryl, heteroarylalkyl, alkenyl, alkenylalkyl, alkynyl, alkynylalkyl, acyl, aroyl, heteroaroyl, aminocarbonyl, and alkoxycarbonyl, all optionally substituted with hydroxy, alkoxy, alkyl, haloalkyl, haloalkoxy, amino, alkylamino, dialkylamino, acylamino, alkylthio, alkylsulfoxyl, alkylsulfonyl, cyano, nitro, and/or halogen. In addition one of the hydroxyl or amino groups of the 2-amino-2,7-dideoxy-alpha-D-glycero-D-gluco-heptopyranosyl moiety can be replaced with a substituent selected from the group consisting of halogen, S(O)ₙR, NR₁R₂, OR, Oacyl, CR₁R₂R₃, CH₂OR, CN, CO₂R, CONR₁R₂, wherein R, R₁, R₂, R₃ are independently selected from the group consisting of H, alkyl, cycloalkyl, alkoxyalkyl, haloalkyl, arylalkyl, and heteroarylalkyl, and n=0-2. In particular, the 6-OH group may be so replaced. Also, the 2-amino group of the 2-amino-2,7-dideoxy-alpha-D-glycero-D-gluco-heptopyranosyl moiety can be functionalized with an acyl group, which may comprise a fatty acid. The 2-amino group may also be alkylated, for example methylated.

The composition of the present invention can further comprise a pharmaceutically acceptable carrier, said carrier being free of components that bind to ribosomal RNA and inhibit translation of envelope virus proteins, and/or assembly or release of viral particles. The aminoglycoside moiety is typically present in a concentration from about 0.001% to about 40% by weight, and may further comprise at least one additive selected from the group consisting of an antimicrobial agent, stabilizer, antifungal agent, analgesic, antioxidant, buffering agent, sunscreen, cosmetic agent, fragrance, lubricant, oil, moisturizer, alcohol, drying agent, preservative, emulsifier, thickening agent, detergent, plasticizer, penetration enhancer, or a mixture thereof. The composition may also further comprise at least one additional antiviral agent, selected from the group consisting of interferon, ribavarin and iminosugars.

Yet another embodiment of the present invention is a method of treating positive sense single-stranded RNA envelope viral infections in a multicellular organism, comprising administering a composition comprising the aminoglycoside moiety 2-amino-2,7-dideoxy-alpha-D-glycero-D-gluco-heptopyranose or an analog thereof, and a pharmaceutically acceptable carrier, to said organism infected with a positive sense single-stranded RNA envelope virus. The aminoglycoside of the composition reduces the infectivity of virus particles. The composition of the method is administered at least once per day over a time period comprising at least one day.

A further embodiment of the present invention is a method for treating positive sense single-stranded RNA envelope viral infections in a multicellular organism, comprising first diagnosing clinical symptoms of the presence of the positive sense single-stranded RNA envelope virus in said organism, followed by administering to said organism a composition comprising the aminoglycoside moiety 2-amino-2,7-dideoxy-alpha-D-glycero-D-gluco-heptopyranose or an analog thereof, and a pharmaceutically acceptable carrier. Clinical symptoms may include the detectable presence of viral antibodies in body fluids, diagnostic levels of viral titer in body fluids, pain, swelling, burning, inflammation, redness, tingling, itching, skin lesions, or a combination thereof.

Administration of the composition may be topical, oral, sublingual, mucosal, trans-membranous, subcutaneous, intravenous, intramuscular, buccal, parentarel, vaginal, anal, transdermal, intracerebroventricular, via ionophoresis, or a combination thereof.

Yet another embodiment of the present invention is a method for preventing the spread of positive sense single-stranded RNA envelope viral infections comprising administering a composition comprising the aminoglycoside moiety 2-amino-2,7-dideoxy-alpha-D-glycero-D-gluco-heptopyranose or an analog thereof, and a pharmaceutically acceptable carrier, in a physiologically appropriate manner to the organism infected with a positive sense single-stranded RNA envelope virus. The aminoglycoside of the composition reduces the infectivity of virus particles.

### BRIEF DESCRIPTION OF THE DRAWINGS

FIG. 1 shows that geneticin protects against NADL-mediated cytotoxicity at 72 hours post infection. (Top panel) geneticin (31-250 μg/ml) has no effect on MDBK cell viability. (Bottom panel) geneticin (1.5-25 μg/ml) treatment improves the viability of infected cells compared to untreated infected cells.

FIG. 2 shows that geneticin inhibits viral load in MDBK cells infected with the NADL or NY-1 strain of BVDV. Panel A) Effect of geneticin, at 6, 12 and 25 μg/ml, on active viral titers of NADL at 24 and 48 hours post infection. Panel B) Effect of geneticin, at 6, 12 and 25 μg/ml, on active viral titers of NY-1 at 24 and 48 hours post infection.

FIG. 3 shows geneticin-mediated cytoprotection against NADL, compared to kanamycin and gentamicin. Panel A) Structural diagrams of kanamycin, gentamicin and geneticin used in this study. Panel B) Only geneticin offers cytoprotection against the NADL strain of BVDV in MDBK cells (all treatments at 12 μg/ml per drug). Interferon is used as a positive control at 100 IU.

FIG. 4 shows that geneticin has no effect on viral translation and processing of NS3 or RNA replication. Panel A shows a Western blot analysis using the primary antibody (Mab 20.10.6) for NS3. Panel B shows the reverse transcriptase-quantitative PCR analysis of intracellular viral RNA in MDBK cells infected with the NADL strain of BVDV in the presence of 6 and 12 μg/ml of geneticin.

### DETAILED DESCRIPTION OF THE INVENTION

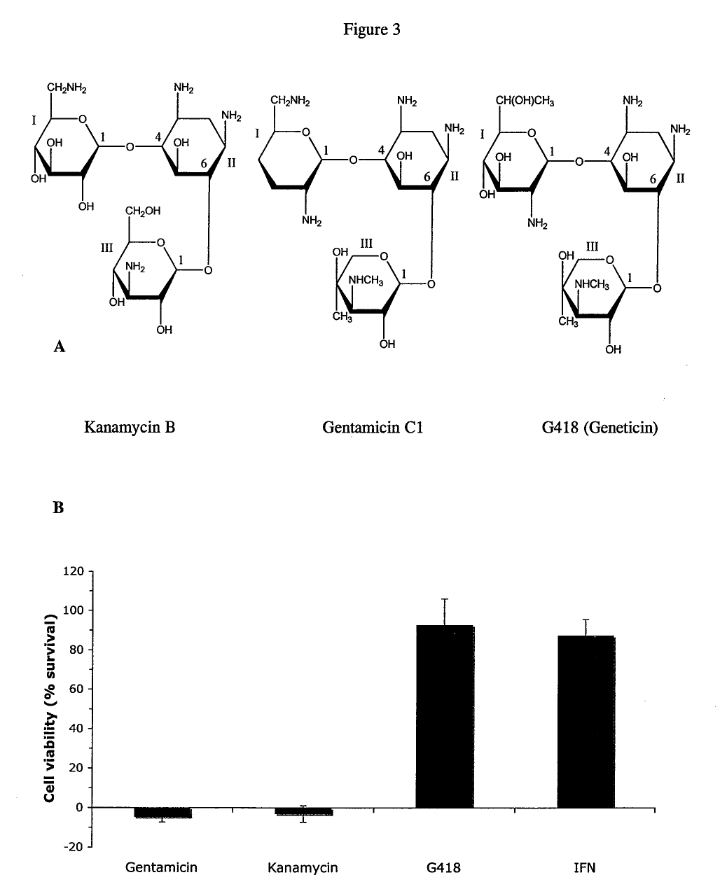

Geneticin (G418, FIG. 3A) is an aminoglycoside antibiotic produced by the Gram-positive soil bacterium Micromonospora rhodorangea. Aminoglycosides are modified sugars, with an amino group replacing one of the sugar hydroxyl groups. Geneticin has been proposed for human use as an anti-parasitic agent (9). In addition, administration of geneticin (10) or the related gentamicin (11) has proven helpful in the treatment of patients suffering from genetic disorders (12).

Geneticin is a 2-amino-2,7-dideoxy-alpha-D-glycero-D-gluco-heptopyranosyl-containing analog of neomycin, widely used to select for transfected eukaryotic cells (13, 14). Geneticin is also used for transfection of stable viral replicons of Flaviviridae family viruses; however, there has been no prior information regarding the effects of geneticin on these RNA viruses.

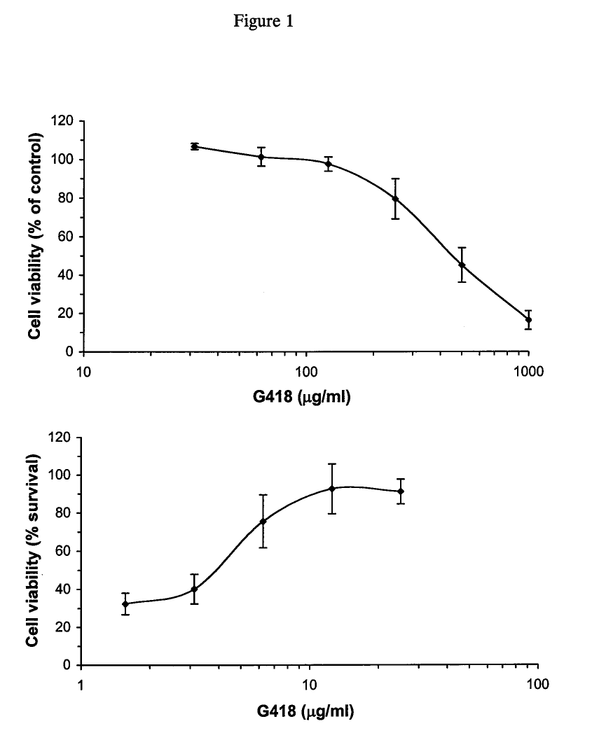

The present invention demonstrates that geneticin is indeed an antiviral agent as evidenced by protection of MDBK cells against the cytopathic effects of the cpBVDV (NADL) and inhibition of production of both cpBVDV (NADL) and ncpBVDV (NY-1), with an EC₅₀ of about 4 μg/ml. The antiviral effects of geneticin are concentration-dependent (FIG. 1). Its EC₅₀ concentration for antiBVDV activity is 4 μg/ml (Table 1), and, at 12.5 μg/ml, it increases cell viability to the level of uninfected control cells. This antiviral and cytoprotective activity of geneticin could be compared with those of interferon, which inhibits BVDV-induced CPE at 100 IU, which is the EC₉₀ for the antiviral activity of interferon in the BVDV system [20,21]. At high doses such as above 400 μg/ml, geneticin was shown to have cell toxicity as reflected by a downward curve of cell viability at those concentrations. The present invention also demonstrates that geneticin blocks monolayer disruption associated with NADL-mediated cytopathology. This antiviral activity of geneticin can be observed out to 72-120 hours post infection, suggesting that viral spread is inhibited.

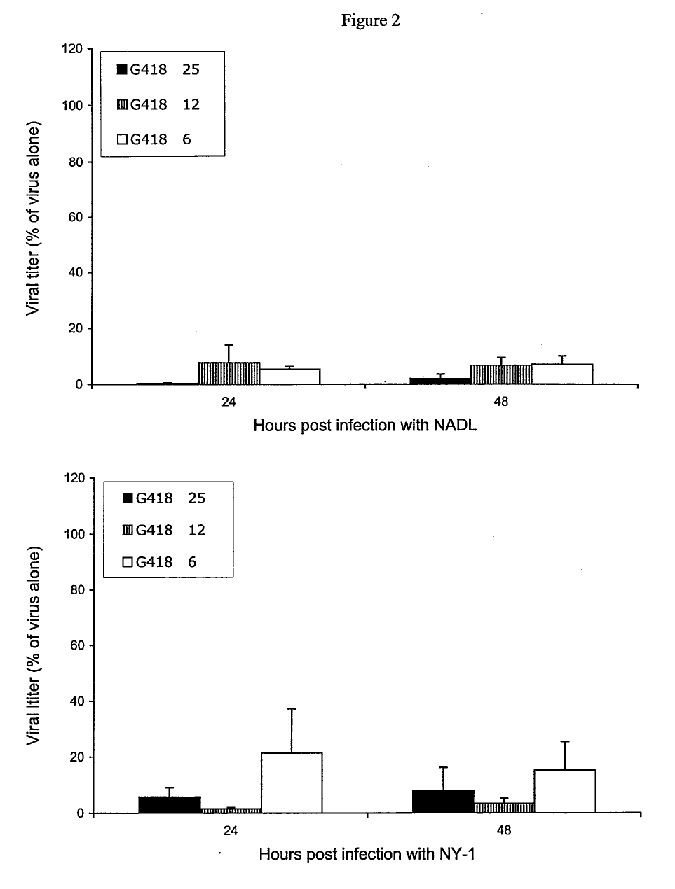

Further characterization of viral replication, by measuring infectious virus released into the cell media, demonstrated that at 24 and 48 hours post infection, geneticin (6-25 μg/ml) inhibited virus yield of both the NADL and NY-1 strains of BVDV by nearly 10-100 fold (FIG. 2). Geneticin clearly reduced viral production to levels below 10% of control during the first 48 h of infection. While not wishing to be bound by any particular theory, in light of the fact that the virus titer method used in our study only measures release of active infectious particles into the media, one may conclude that geneticin either inhibits virus release or reduces the infectivity of virus particles.

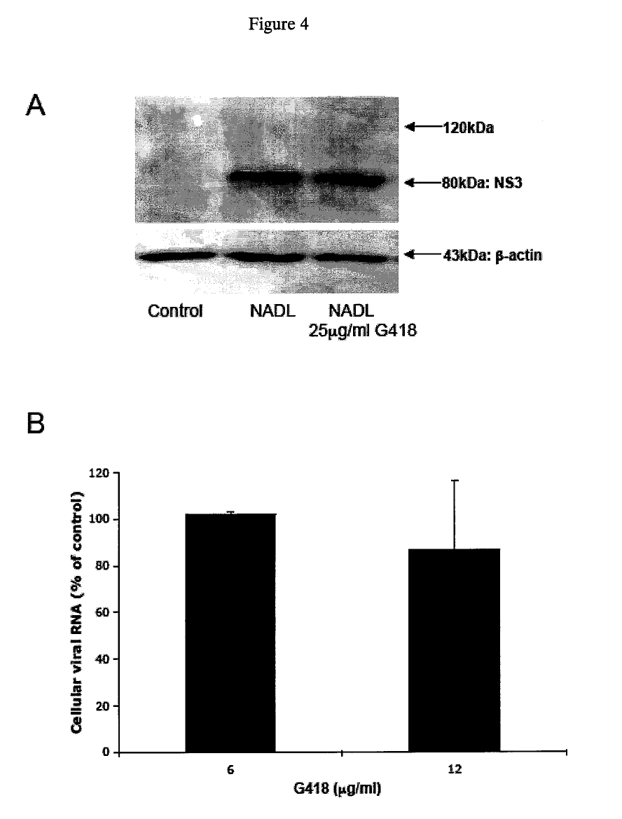

Geneticin at a concentration of 25 μg/ml has no effect on the synthesis of the viral protein NS3, a marker for BVDV translation, at 24 hours post infection with a multiplicity of infection (MOI) of 10 (FIG. 4). A 24 h time point was used to demonstrate stable BVDV translation because at a high MOI (for example, an MOI value of 10) and later time points, viral cytopathology and cell death takes place, making analysis unreliable [14]. In addition, geneticin does not block processing of NS2-NS3 protein (FIG. 4), which is commonly observed for noncytopathic strains of BVDV, demonstrating that geneticin-mediated cytoprotection was not due to inhibition of processing of viral proteins. Also, the 120 kDa band of NS2-NS3, commonly observed during the infection with ncpBVDV [19], could not be detected in geneticin-treated or untreated cells infected with NADL. These data demonstrate that geneticin, although being an inhibitor of protein synthesis (15, 16), does not inhibit virus yield by inhibiting translation or replication directly. In addition, because virus entry precedes translation, it is highly unlikely that geneticin blocks viral mechanisms before translation. Furthermore, assuming that translation is proportional to the amount of RNA, geneticin is unlikely to block RNA transcription. Thus, geneticin controls either assembly or release of active virus, or it reduces the infectivity of virus particles. This conclusion is also supported by plaque assays where even 6 and 12 μg/ml of geneticin significantly reduced plaque size without affecting the number of plaques. Aminoglycosides are thought to inhibit viral replication by interfering with virus entry as well as by inhibiting translation of viral RNAs (17-19); however, the antiviral action of geneticin against BVDV is uniquely different because its mechanism appears to be associated with events subsequent to viral translation, processing and replication.

To determine if geneticin regulates viral RNA replication, intracellular accumulation of viral RNA was measured using RT-qPCR. At 24 h post-infection with BVDV (MOI of 5), there was no difference in the quantities of intracellular viral RNA in the absence or presence of different concentrations of geneticin. Furthermore, the inability of antiviral concentrations of geneticin to inhibit intracellular accumulation of viral RNA at 24 h post-infection also suggests that viral NS5B, required for viral RNA synthesis, was successfully translated and processed. Therefore viral RNA replication is not regulated by geneticin.

Table 1 shows a summary of the antiviral activity of geneticin. The Cytopathic Effect Assay (CPEA) is a measure of cell death induced by the virus; the value generated is an effective concentration of the drug that protects 50% of the cells from virus-induced death (EC₅₀). The Yield Reduction Assay is a measure of the number of active infectious virus particles released from infected cells; the value generated is an effective concentration of the drug that decreases the viral titer by 50% (EC₅₀). The Cytotoxic Concentration required to kill 50% of the cells is defined as the CC₅₀, and is a measure of the intrinsic toxicity of the drug. While not wishing to be bound by any particular theory, the present data indicate that geneticin, and potentially other antiviral agents containing the 2-amino-2,7-dideoxy-alpha-D-glycero-D-gluco-heptopyranosyl moiety, reduces the infectivity of viral particles, thereby inhibiting the spread of the virus and ameliorating symptoms of the viral infection.

TABLE 1
Summary of Antiviral Activity of Geneticin Against Flavi and
Pesti Viruses

| | | CPEA^{c} | YRA^{d} | |
|---|---|---|---|---|
| Virus Name^{a} | Cell^{b} | EC₅₀ μg/ml | EC₅₀ μg/ml | CC₅₀ μg/ml^{e} |
| DV | BHK | 4 | 5 | 160 |
| YFV | BND | 20 | 10 | >2000 |
| BVDV | MDBK | 4 | 4 | 400 |
| BVDV | BND | 50 | nd^{f} | >2000 |

^{a}DV = Dengue Virus, YFV = Yellow Fever Virus, BVDV = Bovine Viral Diarrhea Virus
^{b}BHK = Baby Hamster Kidney, BND = Bottle Nose Dolphin skin cells, MDBK = Madin-Darby Bovine Kidney
^{c}CPEA = Cytopathic Effect Assay, assaying for cell death; EC₅₀ = effective concentration of drug that protects 50% of cells from virus-induced death
^{d}YRA = Yield Reduction Assay, assaying for the reduction in the number of active infectious virus particles released from infected cells; EC₅₀ = effective concentration of drug that causes decrease in viral titer by 50%
^{e}CC₅₀ = Cytotoxic concentration of drug that kills 50% of cells
^{f}nd = not determined

Genetecin has the potential for irreversible ototoxicity and nephrotoxicity, as do most aminoglycosides. New analogs of lower toxicity would greatly aid in the treatment and/or prevention of positive sense single-stranded RNA envelope virus infections.

### Structure-Function Analysis: the Active Moiety

Structure-function analysis of geneticin, as compared to its close structural analogs, kanamycin B and gentamicin, reveals that only geneticin has activity against cpBVDV as demonstrated by significantly improved cell viability of infected cells (FIG. 3). Furthermore, both gentamicin and kanamycin decreased the cell viability of infected cells at the tested concentration. Structurally all three compounds have a common ring II (2-deoxystreptamine), suggesting that the positive sense single-stranded RNA envelope antiviral activity of geneticin is not related to ring II. In addition, gentamicin and geneticin are homologous with regard to ring III, thereby eliminating it as the antiviral moiety. Therefore ring 1,2-amino-2,7-dideoxy-alpha-D-glycero-D-gluco-heptopyranose (Formula I), appears to determine the specificity of geneticin (FIG. 3A) as a positive sense single-stranded RNA envelope antiviral agent. As confirmation, other related aminoglycosides which do not contain the 2-amino-2,7-dideoxy-alpha-D-glycero-D-gluco-heptopyranosyl moiety, such as paromomycin, have no antiviral activity against BVDV. In addition, structurally unrelated antibiotics, for example, tetracycline and fusidic acid, are inactive against BVDV. This structure-function analysis differs from that of aminoglycoside inhibition of protein synthesis via binding to the A site of the 30S ribosome (20), for which the specificity of geneticin binding is correlated with the presence of ring II (FIG. 3A) (21).

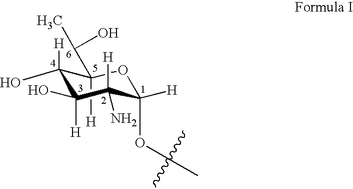

Regarding the chemical structure of Ring I, the 2-amino-2,7-dideoxy-alpha-D-glycero-D-gluco-heptopyranosyl moiety of geneticin, carbon-3 and carbon-4 of the pyranose ring (Formula I and FIG. 3A) are substituted with hydroxyl groups and are identical in both substitution and configuration to those of Ring I of kanamycin (FIG. 3A), which has no antiviral activity. However, for geneticin the substitution on carbon-6 of the 2-amino-2,7-dideoxy-alpha-D-glycero-D-gluco-heptopyranosyl moiety is different from Ring I of kanamycin, with the alcohol being a secondary alcohol by virtue of the additional methyl group substitution of carbon-6. Thus, the structurally relevant difference between these two compounds is related to the functionalization of carbon-6, the carbon atom external to the pyranose ring. The combination of secondary hydroxyl plus methyl substitution at position-6 gives geneticin the lowest affinity to the A site of the small ribosomal subunit (Mingeot-Leclercq et al. 1999; Pfister P S. et al 2005). These observations indicate that the structural requirements for the antiviral activity of geneticin are different from those which determine the inhibition of translation, and support the conclusion that the 2-amino-2,7-dideoxy-alpha-D-glycero-D-gluco-heptopyranosyl moiety is responsible for the positive sense single-stranded RNA envelope antiviral activity of geneticin.

One embodiment of the present invention provides antiviral compounds represented by Formula II, below,

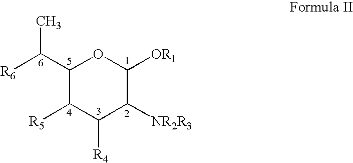

wherein R₁ comprises H, cycloalkyl, carbohydrate, peptide, or nucleotide groups
wherein R₂ and R₃ each independently comprise H, alkyl, cycloalkyl, hydroxyalkyl, alkoxyalkyl, haloalkyl, aryl, arylalkyl, heteroaryl, heteroarylalkyl, alkenyl, alkenylalkyl, alkynyl, alkynylalkyl, acyl, aroyl, heteroaroyl, aminocarbonyl, and alkoxycarbonyl, all optionally substituted with hydroxy, alkoxy, alkyl, haloalkyl, haloalkoxy, amino, alkylamino, dialkylamino, acylamino, alkylthio, alkylsulfoxyl, alkylsulfonyl, cyano, nitro, and/or halogen
wherein R₄, R₅, and R₆ each independently comprise OR₂, halogen, S(O)ₙR₈, CR₉R₁₀R₁₁, CH₂OR, CN, CO₂R, CONR₁₂R₁₃, and NR₁₂R₁₃, wherein R and R₈-R₁₃ are independently selected from the group consisting of H, alkyl, cycloalkyl, alkoxyalkyl, haloalkyl, arylalkyl, heteroarylalkyl, and n=0-2
with the proviso that when R₂ and R₃=H, and R₄, R₅ and R₆=OH, R₁ is other than H.

Preferred compounds of the present invention include those represented by Formula II wherein R₁ is selected from the group consisting of hydrogen and the cyclohexane analogs streptamine and 2-deoxystreptamine; R₁ and R₃ are hydrogen, and R₄, R₅, and R₆ are OH.

As herein described the term “lower alkyl” comprises C₁-C₆ alkyl, branched or linear. The term “cycloalkyl” comprises C₃-C₁₀ cyclic alkyl, for example cyclopropyl, cyclohexyl and cyclodecyl. The term “fatty acid” comprises C₄-C₂₈ carboxylic acids, saturated or unsaturated, linear or branched.

### Compositions

Another embodiment of the present invention provides compositions comprising or consisting essentially of an antiviral agent, which comprises the 2-amino-2,7-dideoxy-alpha-D-glycero-D-gluco-heptopyranosyl moiety and/or its analogs that can inhibit positive sense single-stranded RNA envelope viral spread and infectivity, in a pharmaceutically acceptable carrier, that, when applied in a physiologically appropriate manner to a multicellular organism, such as a human or an animal, lowers the titer of RNA viruses in said organism and prevents or ameliorates the occurrence of infection-related symptoms in the organism.

The antiviral agent of the present invention may comprise 2-amino-2,7-dideoxy-alpha-D-glycero-D-gluco-heptopyranose itself or an analog thereof. The 2-amino-2,7-dideoxy-alpha-D-glycero-D-gluco-heptopyranosyl moiety may be modified as described above, or unmodified. A preferred antiviral agent is geneticin.

The antiviral agent of the composition is present in a concentration of about 0.001% to about 40% by weight, preferably about 0.1% to about 1% by weight. The composition may also further comprise one or more additives, for example an antimicrobial agent, antiviral agent, antifungal agent, analgesic, antioxidant, buffering agent, sunscreen, cosmetic agent, fragrance, lubricant, oil, moisturizer, alcohol, drying agent, preservative, emulsifier, thickening agent, detergent, plasticizer, penetration enhancer, or a mixture thereof. In addition, the pharmaceutically acceptable carrier should be free of components that bind to ribosomal RNA and inhibit translation of envelope virus proteins, and/or assembly or release of viral particles.

Various carriers may be used, so long as they are compatible with the blood and tissues of the human or animal such that the composition may be injected or absorbed without causing deleterious physiological effects or interfere with the function of the active ingredient. For water-soluble antiviral agents, the carrier may be water.

In addition, the compositions may further comprise additional antiviral agents. In particular, antiviral agents that inhibit virus replication by mechanisms of action different from that of geneticin and its analogs, when combined with antiviral agents of the present invention, may amplify the antiviral response and allow reduced dosing, thereby reducing toxicity and minimizing undesirable side effects. Also, such a combination of antiviral agents with different mechanisms of action may serve to reduce the propensity for the virus to develop resistance. Examples of such additional antiviral agents include interferon, ribavarin and iminosugars.

One representative injectable embodiment of the present invention comprises 2-amino-2,7-dideoxy-alpha-D-glycero-D-gluco-heptopyranose and/or its analogs in concentrations from about 0.01 mg/mL to about 500 mg/mL, suspended in a carrier solution of isotonic sodium chloride containing a suitable preservative, such as about 0.1 to about 1.5% benzyl alcohol, stabilizers such as from about 0.25 to about 1% carboxymethylcellulose sodium and about 0.005 to about 0.1% polysorbate 80, plus sufficient sodium hydroxide or hydrochloric acid to adjust the pH to between about 5.0 and about 7.5 (all percentages by weight).

### Viruses

The 2-amino-2,7-dideoxy-alpha-D-glycero-D-gluco-heptopyranosyl-containing antiviral agents of the present invention are selective for positive sense single-stranded RNA envelope viruses. Negative sense single-stranded RNA envelope viruses, for example, influenza viruses, as well as double-stranded DNA viruses, for example, Herpes Simplex virus (HSV) and Hepatitis B virus (HBV) are not affected. Representative positive sense single-stranded RNA envelope viruses include Hepatitis C virus (HCV), West Nile virus (WNV), Yellow Fever virus (YFV), Dengue virus (DV), Bovine Viral Diarrhea virus (BVDV), Equine Arteritis virus (EAV), and Sindbis virus (SINV).

One family of positive sense single-stranded RNA envelope viruses, the Flaviviridae viruses, have monopartite, linear, single-stranded RNA genomes of positive polarity, 9.6- to 12.3-kilobases in length. Virus particles are enveloped and spherical. There are several genera in the family, including the genus Flavivirus, with the species Yellow Fever virus, West Nile virus and Dengue Fever virus; the genus Hepacivirus, with the sole species being Hepatitis C virus; and the genus Pestivirus, including the species Bovine Viral Diarrhea virus and Equine Arteritis virus (EAV), among others that infect non-human mammals. Major diseases caused by the Flaviviridae family include Dengue fever, St. Louis encephalitis, West Nile encephalitis, Yellow fever, and Hepatitis C.

Sindbis virus (SINV) is a member of the Togaviridae family, in the alphavirus subfamily. The virus is transmitted by mosquitoes and causes sindbis fever in humans with symptoms including arthralgia, rash and malaise.

### Analogs

With regard to ring I of geneticin, the 2-amino-2,7-dideoxy-alpha-D-glycero-D-gluco-heptopyranosyl moiety (Formula I; also Formula II where R₁, R₂ and R₃=H, and R₄, R₅, and R₆=OH) can be functionalized with various modifying groups to form derivatives which are also active as antiviral agents of the present invention. For example, the hydroxyl groups attached to carbons-3, -4, and/or -6 may be functionalized to form derivatives. In non-limiting examples, the hydroxyls may be alkylated with alkyl halides or other alkylating agents to form alkoxy groups, using methods known to those skilled in the art. Alternatively the hydroxyls may be acylated to form acyloxy groups using acylating agents such as acid chlorides or anhydrides, for example acetic anhydride. Similarly, the amino group attached to carbon-2 may be alkylated or acylated to form alkylamino and acylamino groups, respectively. For example, the acyl group may be a fatty acid, with a carbon range of 4-28; and the alkyl group may be lower alkyl with a carbon range of 1-6, such as methyl, ethyl, n-butyl or n-hexyl. Additional modifying or functionalizing groups include alkyl, cycloalkyl, hydroxyalkyl, alkoxyalkyl, haloalkyl, aryl, arylalkyl, heteroaryl, heteroarylalkyl, alkenyl, alkenylalkyl, alkynyl, alkynylalkyl, acyl, aroyl, heteroaroyl, aminocarbonyl, and alkoxycarbonyl, all optionally substituted with hydroxy, alkoxy, alkyl, haloalkyl, haloalkoxy, amino, alkylamino, dialkylamino, acylamino, alkylthio, alkylsulfoxyl, alkylsulfonyl, cyano, nitro, and/or halogen.

As used herein, the term “analog” encompasses the derivatives described above, wherein the 2-amino-2,7-dideoxy-alpha-D-glycero-D-gluco-heptopyranosyl moiety has been substituted or functionalized with various modifying groups. The term also encompasses the molecules described below in which one or more of the functional groups of the core molecule, for example, a hydroxyl or amino group, has been replaced with other atoms or groups. In addition the term “analog” also encompasses those conjugates of 2-amino-2,7-dideoxy-alpha-D-glycero-D-gluco-heptopyranose with other biologically relevant molecules, for example, peptides and other carbohydrates, as described below.

With regard to isosteric replacement chemistry, the hydroxyl and/or amino groups of the 2-amino-2,7-dideoxy-alpha-D-glycero-D-gluco-heptopyranosyl moiety can be replaced with, for example halogens, such as fluorine and chlorine. Other hydroxyl and/or amino replacements can include S(O)ₙR, OR, Oacyl, CR₁R₂R₃, CH₂OR, CN, CO₂R, and CONR₁R₂, wherein R, R₁, R₂, R₃ are independently selected from the group consisting of H, alkyl, cycloalkyl, alkoxyalkyl, haloalkyl, arylalkyl, and heteroarylalkyl, and n=0-2, The synthesis methods are known to those skilled in the art.

A particularly useful area for modification is the carbon-6 hydroxyl group, external to the pyranose ring. Compounds of Formula III, are derivatives of the hydroxyl group, as discussed above. Compounds of Formula IV, below, are analogs wherein the 6-hydroxyl group has been replaced by another functional group (X), as discussed above.

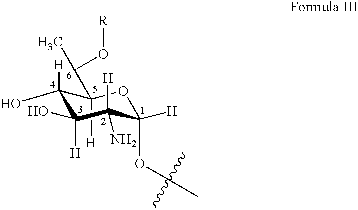

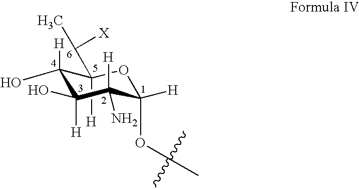

Conjugates of 2-amino-2,7-dideoxy-alpha-D-glycero-D-gluco-heptopyranose with carbohydrates, fatty acids, peptides, or nucleic acids, and their derivatives, are also useful in the treatment of positive sense single-stranded RNA envelope virus infections in multicellular organisms, particularly humans, reducing the infectivity of viral particles, thereby inhibiting the spread of the virus and ameliorating symptoms of the viral infection. The conjugates of either the modified or unmodified 2-amino-2,7-dideoxy-alpha-D-glycero-D-gluco-heptopyranosyl moiety can include, without limitation, carbohydrates, other aminosugars, aminosugar isosteres, for example, streptamine and 2-deoxystreptamine, fatty acids, peptides, and/or nucleic acids, and their derivatives and analogs.

The structural modifications described hereinabove influence the lipophilic/hydrophilic balance (also known as Log P or Log K_{ow}), water solubility, polarity, and other physicochemical properties directly related to drug absorption and distribution. Such modifications may improve the efficacy of the antiviral agents of the present invention. They may also serve to moderate potential toxicity.

### Methods of Treatment

In another embodiment, the present invention provides a method of treating positive sense single-stranded RNA envelope viral infections in multicellular organisms, particularly humans, comprising administering a composition in a physiologically appropriate manner to the organism infected with an RNA envelope virus, wherein the composition comprises at least one antiviral agent comprising the aminoglycoside moiety 2-amino-2,7-dideoxy-alpha-D-glycero-D-gluco-heptopyranose or an analog thereof, that can inhibit positive sense single-stranded RNA envelope viral assembly, release and/or spread, and a pharmaceutically acceptable carrier. A preferred antiviral agent comprises geneticin or an analog thereof. The antiviral effects are achieved by reducing the infectivity of virus particles. Such treatment may act to prevent an initial infection, slow an existing infectious process, or ameliorate the symptoms of an existing positive sense single-stranded RNA envelope virus infection or viral outbreak episode.

The antiviral agent is present in a concentration of about 0.001% to about 40% by weight, preferably about 0.1% to about 1% by weight. The composition is administered at least once per day, and over a time period comprising at least one day.

The method of treatment may also include a preliminary step of diagnosing clinical symptoms of the positive sense single-stranded RNA envelope virus in the organism. These clinical symptoms include the detectable presence of viral antibodies in body fluids, diagnostic levels of viral titer in body fluids, pain, swelling, burning, inflammation, redness, tingling, itching, skin lesions, or a combination thereof. The method of treating positive sense single-stranded RNA envelope viral infections may also comprise the use of a composition further comprising an additive, for example, an antimicrobial agent, antiviral agent, antifungal agent, analgesic, antioxidant, buffering agent, sunscreen, cosmetic agent, fragrance, lubricant, oil, moisturizer, alcohol, drying agent, preservative, emulsifier, thickening agent, detergent, plasticizer, penetration enhancer, or a mixture thereof.

In addition, the method of treating positive sense single-stranded RNA envelope viral infections may also comprise the use of a composition further comprising additional antiviral agents. In particular, antiviral agents that inhibit virus replication by mechanisms of action different from that of geneticin and its analogs, when combined with antiviral agents of the present invention, may allow reduced dosing, thereby reducing toxicity and minimizing undesirable side effects. Also, such a combination of antiviral agents with different mechanisms of action may serve to reduce the propensity for the virus to develop resistance. Examples of such additional antiviral agents include interferon, ribavarin and iminosugars.

Administration of the compositions may be topical, oral, sublingual, mucosal, trans-membranous, subcutaneous, intravenous, intramuscular, buccal, parentarel, vaginal, anal, transdermal, intracerebroventricular, via ionophoresis, or a combination thereof.

Another embodiment of the present invention provides methods of preventing the spread of positive sense single-stranded RNA envelope viral infections. The method comprises administering a composition in a physiologically appropriate manner to the multicellular organism infected with an RNA envelope virus, wherein the composition comprises at least one antiviral agent containing the aminoglycoside moiety 2-amino-2,7-dideoxy-alpha-D-glycero-D-gluco-heptopyranose or an analog thereof, that can inhibit viral spread, and a pharmaceutically acceptable carrier. Viral spread is inhibited by reducing the infectivity of virus particles. A preferred antiviral agent comprises geneticin or an analog thereof.

### EXAMPLES

### Example 1

### Materials and Methods

### Chemicals

All cell culture supplies were obtained from Invitrogen (Carlsbad, Calif., USA). Unless specified, all other reagents were supplied by Sigma Aldrich (St. Louis, Mo., USA).

### Cells and Viruses

MDBK cells (ATCC-CCL22) were grown in Dulbecco's modified Eagle's medium (DMEM) containing 4.5 g of glucose and 10% horse serum, or in Minimum Essential Medium (MEM) with 10% irradiated fetal bovine serum free of antibodies to BVDV. Monolayers of 50-70% confluent cells were infected with plaque-purified cpBVDV strain NADL or ncpBVDV strain NY-1 in cell culture medium, or mock infected with cell culture medium alone. The titre of cpBVDV used in our studies was sufficient to generate an input multiplicity of infection (MOI) of 0.1-0.5. After an initial incubation with virus for 1 h in a cell culture incubator (5% CO₂, 37° C.), the culture medium was changed to a fresh virus-free medium. For aminoglycosides or interferon experiments, drugs were added in required final concentrations to 50-70% confluent cell monolayers and then grown for the next 24-72 h. Interferon was used as a positive antiviral control.

### Cell Viability

MDBK cells were plated at a density 1-2×10³ cells/well in 96-well plates. On the following day after initial infection for 1 h, cells were washed and medium containing various concentrations of inhibitors were added to cells and incubated for the desired period of time. Cell viability was assessed using the resaruzin (almarBlue) indicator dye [10]. Quantitative analysis of dye conversion (AFU) was measured using a fluorescent plate reader with excitation/emission=550/580 nm. For toxicity studies, cell viability was expressed as a percent of control (AFU_{treated}/AFU_{control}). The drug mediated protection of cell viability was expressed as percent survival as follows: % survival=[(AFU_{treated})BVDV−(AFU_{control})BVDV]/[(AFU_{control})mock−(AFU_{control})BVDV], where (AFU_{treated})BVDV is the AFU of cells infected with BVDV and treated with a certain dilution of geneticin, (AFU_{control})BVDV is the AFU of cells infected with BVDV and left untreated, and (AFU_{control})mock is the AFU of cells mock infected and left untreated. The 50% effective concentration (EC₅₀) was defined as the concentration of compound that offered 50% protection of the cells against virus-induced cytopathic effect and was calculated using logarithmic interpolation [11].

### Plate Viability Assay

MDBK cells were infected with the NADL strain of BVDV at an MOI of 0.1 and distributed to a collagen-coated, 24-well plate. Cells were washed with phosphate-buffered saline (PBS) once after 6 h incubation at 37° C. and 5% CO₂, followed by addition of 2.5% methyl cellulose in the DMEM media containing 5% heat-inactivated horse serum. Crystal violet staining was performed 5 days thereafter.

### Determination of Viral Titres

Each well of a 12-well tissue culture plate was seeded with 1.5×10⁵ MDBK cells and incubated at 37° C. with 5% CO₂ for 24 h. Then, after removing the medium and rinsing the monolayer, cells were infected with BVDV at an MOI of about 1-2 to ensure efficiency of infection. Virus was then absorbed to cells at 4° C. for 1 h and rinsed with cold PBS before the addition of required concentrations of geneticin in growth medium. At 24 and 48 h post-infection, 0.25 ml aliquots of supernatant were harvested from each well and stored at −70° C. beibre further analysis of viral titres. To determine viral titres, 10-fold dilutions (10⁻¹-10⁻⁸) of harvested supernatants were prepared and 50 μl of each dilution was plated into each of eight vertical wells per dilution of 96-well plate containing 50 μl of MDBK cells. Cells were then incubated at 37° C. for 96-120 h and wells were scored for cytopathic effects (CPE) of the virus in order to determine viral titres according to Reed-Muench [12]. For ncpBVDV (NY-1), titre plates were fixed at 120 h with 20% acetone. Presence of virus was detected using BVDV MAb 20.10.6 with an immunoperoxidase test system.

### Western Blot Analysis of Viral NS3 Protein

MDBK cells were infected with NADL strain of BVDV (MOI 10) in the presence or absence of 25 μg/ml of geneticin. Based on previous publications [13], the 18-24 h post-infection time point was chosen to determine viral proteins in infected cells. In addition, it had previously been demonstrated that in MDBK cells at 36 h with this high MOI, cell monolayers deteriorate very rapidly resulting in virus-induced cell death [14]. Thus, at 24 h post-infection, cell media was removed and cells were washed several times with PBS. Then, 2× concentrated electrophoresis sample buffer was added, cells were scraped from the dish and transferred to a microcentrifuge tube. To reduce viscosity, the samples were sonicated briefly or passed several times through a 26-gauge needle and boiled for 5 min. Fifteen micrograms of lysate sample were run on 10% SDS-PAGE and then transblotted. After blocking for 1 h in blocking buffer (5% dry milk in 0.05% Tween 20 in PBS), membranes were incubated with primary antibody (MAb 20.10.6) [15] diluted 1:1,000 in blocking buffer for 60 min at room temperature and washed with 0.05% Tween 20 in PBS. After incubation with secondary antibody (anti-mouse IgG-HRP; Sigma) diluted 1:200 in blocking buffer for 60 min at room temperature, NS3 was detected with the Amersham ECL kit according to the manufacturer's instructions (Amersham Biosciences, Piscataway, N.J., USA).

### Detection of Intracellular Viral RNA by RT-Quantitative PCR

A 24 h post-infection time point was used because overall viral RNA synthesis drops about two- to threefold in infected cells at 48 h post-infection (data not shown). This decrease in virus production in infected cells could be due to rapid cell death observed at this high MOI [14]. Thus, at 24 h post-infection with NADL strain of BVDV (MOI 10) in the presence or absence of 6 and 12 μg/ml of geneticin, medium was removed from the cell monolayers and washed with PBS three times. RNA was isolated using the RNAeasy kit (Qiagen, Valencia, Calif., USA). A 50 μl RT-quantitative PCR (qPCR) was performed according to a previous publication [16] with forward primer corresponding to nucleotides 103-123 (5-TAG CCA TGC CCT TAG TAG GAC-3), reverse primer corresponding to nucleotides 176-196 (5-GAC GAC TAC CCT GTA CTC AGG-3) and TaqMan probe (6-carboxyfluorescein-AAC AGT GGT GAG TTC GTT GGA TGG CTT-6-carboxytetramethylrhodamine). PCR amplification consisted of 40 cycles of denaturation at 94° C. for 20 s and annealing and extension at 62° C. for 1 min in an ABI 7000 sequence detector. All samples were analysed in three replicate reactions.

FIG. 1 shows that geneticin protects against NADL-mediated cytotoxicity at 72 hours post infection. The top panel shows that geneticin (31-250 μg/ml) has no effect on MDBK cell viability. Cell viability is expressed as a percent of cell viability of geneticin-treated cells over control (untreated) cells. The bottom panel shows that geneticin (1.5-25 μg/ml) improves cell viability of infected cells, compared to the untreated virally infected cells. Cell viability is expressed as a percent of cell viability of geneticin-treated NADL-infected cells over untreated infected cells. Cell viability is assessed in 96 well plates, using the Resaruzin (Almar blue) indicator dye (22). Quantitative analysis of dye conversion is measured using a fluorescent plate reader with ex/em=550/580. Error bars indicate the standard error for each concentration of geneticin (n=6).

### Example 2

FIG. 2 show that geneticin inhibits viral load in MDBK cells infected with NADL or NY-1. Panel A shows the effect of geneticin, at 6, 12 and 25 μg/ml, on active viral titers of NADL at 24, 48, and 72 hours post infection. Panel B shows the effect of geneticin, at 6, 12 and 25 μg/ml, on active viral titers of NY-1 at 24, 48, and 72 hours post infection. Viral titers were determined according to Reed-Muench (Spector, S., and Lancz, G. 1986. Clinical Virology Manual. Elsevier Science Pub. Co. N.Y. pp. 194. Snyder, M. L., Stewart, W. C., Kresse, J. I. Microtitration Neutralization Test for PRV and TGE. 1981. Serologic Microtitration Techniques. NVSL, USDA, Ames, Iowa. pp. 44-45), using CPE or NS3 Mab 20.10.6, for NADL or NY-1, respectively. Error bars indicate the standard error for each time point and specified concentration of geneticin (n=3).

### Example 3

FIG. 3 shows geneticin-mediated cytoprotection against NADL, compared to kanamycin and gentamicin. Panel B shows that only geneticin offers cytoprotection against NADL. All aminoglycosides were used at 6, 12, and 25 μg/ml. Cell viability was assessed in 96 well plates, using the Resaruzin (Almar blue) indicator dye (22). Quantitative analysis of dye conversion was measured using a fluorescent plate reader with ex/em=550/580. Error bars indicate the standard error for each aminoglycoside and specified concentration.

### Example 4

FIG. 4 shows that geneticin has no effect on viral translation and processing of NS3. Panel A: MDBK cells were infected with the NADL strain of BVDV in the absence or presence of 25 μg/ml of geneticin, and at 24 h post-infection, membrane and cytosolic fractions were prepared. Samples were subjected to SDS-PAGE and Western blotting analysis using the primary antibody (Mab 20.10.6) for NS3. Panel B shows the reverse transcriptase-quantitative PCR analysis of intracellular viral RNA in MDBK cells infected with the NADL strain of BVDV in the presence of 6 and 12 μg/ml of geneticin, at 24 h post-infection. Viral RNA expression is defined as a percent of viral RNA collected from geneticin-treated infected cells over RNA detected from untreated infected cells. Error bars indicate the standard deviation for each specified condition (n=3).

### Example 5

This example describes an injectable composition of the present invention using geneticin. A solution of geneticin, 0.1-10 mg/kg, is dissolved in a sterile carrier solution of isotonic sodium chloride containing 1.0% benzyl alcohol as preservative, and 1% carboxymethylcellulose sodium and 0.05% polysorbate 80 as stabilizers. The pH is adjusted to 7.0 with sodium hydroxide or hydrochloric acid.

### Example 6

This example describes an injectable composition of the present invention using 2-amino-2,7-dideoxy-alpha-D-glycero-D-gluco-heptopyranose and the additional antiviral agent interferon. A solution of 2-amino-2,7-dideoxy-alpha-D-glycero-D-gluco-heptopyranose, 0.1-10 mg/kg, is dissolved in a sterile carrier solution of isotonic sodium chloride containing 1.0% benzyl alcohol as preservative, and 1% carboxymethylcellulose sodium and 0.05% polysorbate 80 as stabilizers. The pH is adjusted to 7.0 with sodium hydroxide or hydrochloric acid. Interferon, 1000 IU, is added.

### Example 7

This example describes a method of treating a Dengue virus infection according to the present invention. A patient showing symptoms of Dengue Hemorrhagic Fever (DHF) is injected daily for 7 days with the composition of Example 5. The dosage ranges from 0.1-10 mg/kg.

### Example 8

This example describes a method of treating an HCV infection according to the present invention. A patient is diagnosed with clinical symptoms of HCV, including the presence of HCV in the blood. The patient is injected daily for at least 30 days with the composition of Example 6. The dosage of the antiviral agent of the present invention ranges from 0.1-10 mg/kg. The presence of HCV in the blood is monitored. A “sustained response” means that the patient remains free of HCV for 6 months after stopping treatment. This does not mean that the patient is cured, but that the levels of active HCV in the body are very low and are probably not causing as much damage.

Those skilled in the art will recognize that the above examples are illustrative of the present invention and not necessarily limiting thereto. Many other embodiments may be envisioned which are encompassed by the present invention, and the following claims.

### REFERENCES

1. Alter H J, Seeff L B. Recovery, persistence, and sequelae in hepatitis C virus infection: a perspective on long-term outcome. Semin Liver Dis. 2000; 20(1):17-35.
2. Cohen J. The scientific challenge of hepatitis C. Science. 1999 Jul. 2; 285(5424):26-30.
3. Dengue/dengue haemorrhagic fever. Wkly Epidemiol Rec. 2000 Jun. 16; 75(24): 193-6.
4. Collett M S, Anderson D K, Retzel E. Comparisons of the pestivirus bovine viral diarrhoea virus with members of the flaviviridae. J Gen Virol. 1988 October; 69 (Pt 10):2637-43.
5. Brownlie J, Clarke M C, Howard C J. Experimental production of fatal mucosal disease in cattle. Vet Rec. 1984 Jun. 2; 114(22):535-6.
6. Bolin S R, McClurkin A W, Cutlip R C, Coria M F. Severe clinical disease induced in cattle persistently infected with noncytopathic bovine viral diarrhea virus by superinfection with cytopathic bovine viral diarrhea virus. Am J Vet Res. 1985 March; 46(3):573-6.
7. Brownlie J. The pathways for bovine virus diarrhoea virus biotypes in the pathogenesis of disease. Arch Virol Suppl. 1991; 3:79-96.
8. Buckwold V E, Beer B E, Donis R O. Bovine viral diarrhea virus as a surrogate model of hepatitis C virus for the evaluation of antiviral agents. Antiviral Res. 2003 September; 60(1):1-15.
9. Griffiths J K, Balakrishnan R, Widmer G, Tzipori S. Paromomycin and geneticin inhibit intracellular Cryptosporidium parvum without trafficking through the host cell cytoplasm: implications for drug delivery. Infect Immun. 1998 August; 66(8):3874-83.
10. Howard M T, Shirts B H, Petros L M, Flanigan K M, Gesteland R F, Atkins J F. Sequence specificity of aminoglycoside-induced stop condon readthrough: potential implications for treatment of Duchenne muscular dystrophy. Ann Neurol. 2000 August; 48(2): 164-9.
11. Stephenson J. Antibiotics show promise as therapy for genetic disorders. Jama. 2001 Apr. 25; 285(16):2067-8.
12. Barton-Davis E R, Cordier L, Shoturma D I, Leland S E, Sweeney H L. Aminoglycoside antibiotics restore dystrophin function to skeletal muscles of mdx mice. J Clin Invest. 1999 August; 104(4):375-81.
13. Danielson K G, Knepper J E, Kittrell F S, Butel J S, Medina D, Durban E M. Clonal populations of the mouse mammary cell line, COMMA-D, which retain capability of morphogenesis in vivo. In Vitro Cell Dev Biol. 1989 June; 25(6):535-43.
14. Santerre R F, Allen N E, Hobbs J N, Jr., Rao R N, Schmidt R J. Expression of prokaryotic genes for hygromycin B and G418 resistance as dominant-selection markers in mouse L cells. Gene. 1984 October; 30(1-3):147-56.
15. Cabanas M J, Vazquez D, Modolell J. Inhibition of ribosomal translocation by aminoglycoside antibiotics. Biochem Biophys Res Commun. 1978 Aug. 14; 83(3):991-7.
16. Eustice D C, Wilhelm J M. Mechanisms of action of aminoglycoside antibiotics in eucaryotic protein synthesis. Antimicrob Agents Chemother. 1984 July; 26(1):53-60.
17. Langeland N, Haarr L, Holmsen H. Evidence that neomycin inhibits HSV 1 infection of BHK cells. Biochem Biophys Res Commun. 1986 Nov. 26; 141(1):198-203.
18. Langeland N, Oyan A M, Marsden H S, Cross A, Glorioso J C, Moore L J, et al. Localization on the herpes simplex virus type 1 genome of a region encoding proteins involved in adsorption to the cellular receptor. J Virol. 1990 March; 64(3):1271-7.
19. Zapp M L, Stern S, Green M R. Small molecules that selectively block RNA binding of HIV-1 Rev protein inhibit Rev function and viral production. Cell. 1993 Sep. 24; 74(6):969-78.
20. Benveniste R, Davies J. Structure-activity relationships among the aminoglycoside antibiotics: role of hydroxyl and amino groups. Antimicrob Agents Chemother. 1973 October; 4(4):402-9.
21. Vicens Q, Westhof E. Crystal structure of geneticin bound to a bacterial 16S ribosomal RNA A site oligonucleotide. J Mol Biol. 2003 Feb. 28; 326(4):1175-88.
22. Mazzio E A, Soliman K F. Glioma cell antioxidant capacity relative to reactive oxygen species produced by dopamine. J Appl Toxicol. 2004 March-April; 24(2):99-106.

What is claimed is:

1. A compound of Formula II 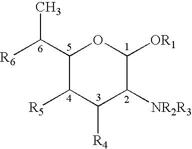
wherein R₁ comprises H, cycloalkyl, carbohydrate, peptide, or nucleotide groups
wherein R₂ and R₃ each independently comprise H, alkyl, cycloalkyl, hydroxyalkyl, alkoxyalkyl, haloalkyl, aryl, arylalkyl, heteroaryl, heteroarylalkyl, alkenyl, alkenylalkyl, alkynyl, alkynylalkyl, acyl, aroyl, heteroaroyl, aminocarbonyl, and alkoxycarbonyl, all optionally substituted with hydroxy, alkoxy, alkyl, haloalkyl, haloalkoxy, amino, alkylamino, dialkylamino, acylamino, alkylthio, alkylsulfoxyl, alkylsulfonyl, cyano, nitro, and/or halogen
wherein R₄, R₅, and R₆ each independently comprise OR₂, halogen, S(O)ₙR₈, CR₉R₁₀R₁₁, CH₂OR, CN, CO₂R, CONR₁₂R₁₃, and NR₁₂R₁₃, wherein R and R₈-R₁₃ are independently selected from the group consisting of H, alkyl, cycloalkyl, alkoxyalkyl, haloalkyl, arylalkyl, heteroarylalkyl, and n=0-2
with the proviso that when R₂ and R₃=H, and R₄, R₅ and R₆=OH, R₁ is other than H.

2. The compound of claim 1, wherein
R₁ is selected from the group consisting of streptamine, or 2-deoxystreptamine
R₂ and R₃ are H
And R₄, R₅, and R₆ are OH.

3. A composition for treating positive sense single-stranded RNA envelope viral infections comprising the aminoglycoside moiety 2-amino-2,7-dideoxy-alpha-D-glycero-D-gluco-heptopyranose, or an analog thereof.

4. The composition of claim 3, wherein said composition reduces the infectivity of virus particles.

5. The composition of claim 3, wherein the viral infection is produced by a positive sense single-stranded RNA envelope virus selected from the group consisting of Hepatitis C virus (HCV), West Nile virus (WNV), Yellow Fever virus (YFV), Dengue virus (DV), Bovine Viral Diarrhea virus (BVDV), Equine Arteritis virus (EAV), and Sindbis virus (SINV), or a combination of said viruses.

6. The composition of claim 3, wherein the aminoglycoside moiety comprises at least one unmodified 2-amino-2,7-dideoxy-alpha-D-glycero-D-gluco-heptopyranosyl moiety.

7. The composition of claim 3, wherein the aminoglycoside moiety comprises at least one conjugate of a 2-amino-2,7-dideoxy-alpha-D-glycero-D-gluco-heptopyranosyl moiety.

8. The composition of claim 3, wherein the aminoglycoside moiety comprises geneticin or an analog thereof.

9. The composition of claim 8, wherein the analog of geneticin is functionalized with at least one modifying group on the 2-amino-2,7-dideoxy-alpha-D-glycero-D-gluco-heptopyranosyl moiety.

10. The composition of claim 3, wherein at least one hydroxyl group of the 2-amino-2,7-dideoxy-alpha-D-glycero-D-gluco-heptopyranosyl moiety is functionalized with a modifying group.

11. The composition of claim 10, wherein the hydroxyl group of carbon-6 is functionalized with a modifying group.

12. The composition of claim 10, wherein the modifying group is selected from the group consisting of alkyl, cycloalkyl, hydroxyalkyl, alkoxyalkyl, haloalkyl, aryl, arylalkyl, heteroaryl, heteroarylalkyl, alkenyl, alkenylalkyl, alkynyl, alkynylalkyl, acyl, aroyl, heteroaroyl, aminocarbonyl, and alkoxycarbonyl, all optionally substituted with hydroxy, alkoxy, alkyl, haloalkyl, haloalkoxy, amino, alkylamino, dialkylamino, acylamino, alkylthio, alkylsulfoxyl, alkylsulfonyl, cyano, nitro, and/or halogen.

13. The composition of claim 11, wherein the modifying group is selected from the group consisting of alkyl, cycloalkyl, hydroxyalkyl, alkoxyalkyl, haloalkyl, aryl, arylalkyl, heteroaryl, heteroarylalkyl, alkenyl, alkenylalkyl, alkynyl, alkynylalkyl, acyl, aroyl, heteroaroyl, aminocarbonyl, and alkoxycarbonyl, all optionally substituted with hydroxy, alkoxy, alkyl, haloalkyl, haloalkoxy, amino, alkylamino, dialkylamino, acylamino, alkylthio, alkylsulfoxyl, alkylsulfonyl, cyano, nitro, and/or halogen.

14. The composition of claim 3, wherein one of the hydroxyl or amino groups of the 2-amino-2,7-dideoxy-alpha-D-glycero-D-gluco-heptopyranosyl moiety is replaced with a substituent selected from the group consisting of halogen, S(O)ₙR, NR₁R₂, OR, Oacyl, CR₁R₂R₃, CH₂OR, CN, CO₂R, CONR₁R₂, wherein R, R₁, R₂, R₃ are independently selected from the group consisting of H, alkyl, cycloalkyl, alkoxyalkyl, haloalkyl, arylalkyl, and heteroarylalkyl, and n=0-2.

15. The composition of claim 14, wherein the hydroxyl group of carbon-6 is replaced with a substituent selected from the group consisting of halogen, S(O)ₙR, NR₁R₂, OR, Oacyl, CR₁R₂R₃, CH₂OR, CN, CO₂R, CONR₁R₂, wherein R, R₁, R₂, R₃ are independently H, alkyl, cycloalkyl, alkoxyalkyl, haloalkyl, arylalkyl, and heteroarylalkyl, and n=0-2.

16. The composition of claim 3, wherein the 2-amino group of the 2-amino-2,7-dideoxy-alpha-D-glycero-D-gluco-heptopyranosyl moiety is functionalized with an acyl group.

17. The composition of claim 16, wherein the acyl group is a fatty acid.

18. The composition of claim 3, wherein the 2-amino group of the 2-amino-2,7-dideoxy-alpha-D-glycero-D-gluco-heptopyranosyl moiety is alkyl substituted.

19. The composition of claim 18 wherein the alkyl group is methyl.

20. The composition of claim 3, wherein said composition further comprises a pharmaceutically acceptable carrier and said carrier is free of components that bind to ribosomal RNA and inhibit translation of envelope virus proteins, and/or assembly or release of viral particles.

21. The composition of claim 3, wherein the aminoglycoside moiety is present in a concentration from about 0.001% to about 40% by weight.

22. The composition of claim 3, wherein the composition further comprises at least one additive.

23. The composition of claim 22, wherein the additive is selected from the group consisting of an antimicrobial agent, stabilizer, antifungal agent, analgesic, antioxidant, buffering agent, sunscreen, cosmetic agent, fragrance, lubricant, oil, moisturizer, alcohol, drying agent, preservative, emulsifier, thickening agent, detergent, plasticizer, penetration enhancer, or a mixture thereof.

24. The composition of claim 3, wherein said composition is used to treat a multicellular organism.

25. A method of treating positive sense single-stranded RNA envelope viral infections in a multicellular organism, comprising administering a composition comprising the aminoglycoside moiety 2-amino-2,7-dideoxy-alpha-D-glycero-D-gluco-heptopyranose or an analog thereof, and a pharmaceutically acceptable carrier, to said organism infected with a positive sense single-stranded RNA envelope virus.

26. The method of claim 25, wherein said composition reduces the infectivity of virus particles.

27. The method of claim 25, wherein said aminoglycoside moiety is present in a concentration of from about 0.001% to about 40% by weight.

28. The method of claim 25, wherein the composition is administered at least once per day.

29. The method of claim 25, wherein the composition is administered to said organism over a time period comprising at least one day.

30. A method for treating positive sense single-stranded RNA envelope viral infections in a multicellular organism, comprising:
a) diagnosing clinical symptoms of the presence of the positive sense single-stranded RNA envelope virus in said organism; and
b) administering to said organism a composition comprising the aminoglycoside moiety 2-amino-2,7-dideoxy-alpha-D-glycero-D-gluco-heptopyranose or an analog thereof, and a pharmaceutically acceptable carrier.

31. The method of claim 30, wherein viral infection is inhibited by reducing the infectivity of virus particles.

32. The method of claim 30, wherein said clinical symptoms comprise the detectable presence of viral antibodies in body fluids, diagnostic levels of viral titer in body fluids, pain, swelling, burning, inflammation, redness, tingling, itching, skin lesions, or a combination thereof.

33. The method of claim 25, wherein the pharmaceutically acceptable carrier further comprises at least one additive.

34. The method of claim 33, wherein said additive is selected from the group consisting of an antimicrobial agent, stabilizer, antifungal agent, analgesic, antioxidant, buffering agent, sunscreen, cosmetic agent, fragrance, lubricant, oil, moisturizer, alcohol, drying agent, preservative, emulsifier, thickening agent, detergent, plasticizer, penetration enhancer, or a mixture thereof.

35. The method of claim 25, wherein administration of the composition may be topical, oral, sublingual, mucosal, trans-membranous, subcutaneous, intravenous, intramuscular, buccal, parentarel, vaginal, anal, transdermal, intracerebroventricular, via ionophoresis, or a combination thereof.

36. The method of claim 30, wherein administration of the composition may be topical, oral, sublingual, mucosal, trans-membranous, subcutaneous, intravenous, intramuscular, buccal, parentarel, vaginal, anal, transdermal, intracerebroventricular, via ionophoresis, or a combination thereof.

37. A method for preventing the spread of positive sense single-stranded RNA envelope viral infections comprising administering a composition comprising the aminoglycoside moiety 2-amino-2,7-dideoxy-alpha-D-glycero-D-gluco-heptopyranose or an analog thereof, and a pharmaceutically acceptable carrier, in a physiologically appropriate manner to the organism infected with a positive sense single-stranded RNA envelope virus.

38. The method of claim 37, wherein viral infection is inhibited by reducing the infectivity of virus particles.

39. The composition of claim 3, further comprising at least one additional antiviral agent.

40. The composition of claim 39, where said antiviral agent is selected from the group consisting of interferon, ribavarin and iminosugars.

41. The composition of claim 8, further comprising at least one additional antiviral agent selected from the group consisting of interferon, ribavarin and iminosugars.

42. The method of claim 25, wherein said composition further comprises at least one additional antiviral agent.

43. The method of claim 42, wherein said antiviral agent is selected from the group consisting of interferon, ribavarin and iminosugars.

44. The method of claim 30, wherein said composition further comprises at least one additional antiviral agent.

45. The method of claim 44, wherein said antiviral agent is selected from the group consisting of interferon, ribavarin and iminosugars.

46. The method of claim 37, wherein said composition further comprises at least one additional antiviral agent.

47. The method of claim 46, wherein said antiviral agent is selected from the group consisting of interferon, ribavarin and iminosugars.
